(19) United States
(12) Patent Application Publication
(10) Pub. No.: US 2018/0317856 A1
(43) Pub. Date: Nov. 8, 2018

(54) COGNITIVE ANALYSIS OF E-TATTOO ACQUIRED PALPATION MEASUREMENTS

(71) Applicant: International Business Machines Corporation, Armonk, NY (US)

(72) Inventors: James E. Bostick, Cedar Park, TX (US); John M. Ganci, JR., Cary, NC (US); Martin G. Keen, Cary, NC (US); Sarbajit K. Rakshit, Kolkata (IN)

(21) Appl. No.: 15/854,619

(22) Filed: Dec. 26, 2017

Related U.S. Application Data
(63) Continuation of application No. 15/589,966, filed on May 8, 2017.

(51) Int. Cl. A61B 5/00; A61B 5/01; G16H 10/60; G16H 50/20; G16H 30/20

(52) U.S. Cl. CPC A61B 5/7246; A61B 5/742; A61B 5/6806; A61B 5/4827; A61B 5/01; A61B 5/0077; A61B 5/0022; A61B 5/002; G16H 50/20; G16H 30/20; G16H 10/60

(57) ABSTRACT

Embodiments of the present invention provide a method, system and computer program product for cognitive analysis of e-tattoo acquired palpation measurements. In an embodiment of the invention, a method of cognitive analysis of e-tattoo acquired palpation measurements includes receiving pressure data from an e-tattoo affixed to surgical gloves in connection with a contemporaneous palpation of a patient and storing the received pressure data in a data store. The method also includes retrieving from the data store previously stored pressure data also received from a different e-tattoo affixed to different surgical gloves in connection with a previous palpation of the patient. Finally, the method includes generating in a display of a computer a visualization of a comparison of the pressure data both received and previously stored.

### BACKGROUND OF THE INVENTION

### Field of the Invention

The present invention relates to palpation data acquisition and more particularly to e-tattoo acquired palpation data.

### Description of the Related Art

Palpation refers to the examination with hands of a patient so as to feel for the condition of a bodily organ, fluid, mass or movement of a bodily organ, fluid or mass. Generally, a health care practitioner utilizes palpation in order to perceive and/or diagnose a particular condition in the body. More specifically, during palpation, the health care provider touches and feels the body in order to examine the size, consistency, texture, location, and tenderness of an organ or body part. Oftentimes, the health care provider concurrently observes the facial reaction of the subject patient during palpation, or the health care provider listens for an audible reaction by the subject. Combining the tactile feedback felt by the hands of the health care provider with the observed reaction of the subject, the health care provider then provides an assessment of the palpation.

Palpation as a diagnostic technique oftentimes requires the comparison of multiple different palpation examinations so as to assess a change in the condition of the portion of interest of the subject. To perform this comparison, the health care provider must maintain a recorded recollection of the past palpation for comparison to the contemporaneously acquired palpation data. With any translation of recorded data to the memory of a particular tactile sensation, of course, data is lost and the process thus can be imprecise. Furthermore, in many instances two different health care providers perform different ones of the palpative examinations so that the second health care provider performing the later palpation lacks first hand knowledge of the outcome of the earlier palpation. Consequently, the efficiency of the examination is lost and the quality of patient care suffers.

### BRIEF SUMMARY OF THE INVENTION

Embodiments of the present invention address deficiencies of the art in respect to palpation driven patient assessment and provide a novel and non-obvious method, system and computer program product for cognitive analysis of e-tattoo acquired palpation measurements. In an embodiment of the invention, a method of cognitive analysis of e-tattoo acquired palpation measurements includes receiving pressure data from an e-tattoo affixed to surgical gloves in connection with a contemporaneous palpation of a patient and storing the received pressure data in a data store. The method also includes retrieving from the data store previously stored pressure data also received from a different e-tattoo affixed to different surgical gloves in connection with a previous palpation of the patient. Finally, the method includes generating in a display of a computer a visualization of a comparison of the pressure data both received and previously stored.

In one aspect of the embodiment, the method additionally includes acquiring video imagery of the patient during the contemporaneous palpation. Thereafter, previously stored video imagery of the patient during the previous palpation is additionally retrieved and presented the display the previously stored video imagery. In another aspect of the embodiment, the method additionally includes acquiring audio of the patient during the contemporaneous palpation. Thereafter, previously stored audio of the patient is additionally retrieved and played back through the computer the previously stored video imagery. In even yet another aspect of the embodiment, the method further includes locating in the data store a diagnostic notation corresponding to the received pressure data during the contemporaneous palpation and displaying in the display the located diagnostic notation.

In another embodiment of the invention, a data processing system is configured for cognitive analysis of e-tattoo acquired palpation measurements. The system includes a host computing system with memory and at least one processor, a display and a communicatively coupled data store. The system also includes a cognitive analysis module executing in the memory of the host computing system. The module includes program code enabled upon execution by the at least one processor to receive pressure data from an e-tattoo affixed to surgical gloves in connection with a contemporaneous palpation of a patient and storing the received pressure data in the data store, to retrieve from the data store previously stored pressure data also received from a different e-tattoo affixed to different surgical gloves in connection with a previous palpation of the patient, and to generate in the display a visualization of a comparison of the pressure data both received and previously stored.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for cognitive analysis of e-tattoo acquired palpation measurements;

FIG. 2 is a schematic illustration of a data processing system configured for cognitive analysis of e-tattoo acquired palpation measurements; and,

FIG. 3 is a flow chart illustrating a process for cognitive analysis of e-tattoo acquired palpation measurements.

### DETAILED DESCRIPTION OF THE INVENTION

Embodiments of the invention provide for cognitive analysis of e-tattoo acquired palpation measurements. In accordance with an embodiment of the invention, an e-tattoo is configured with a pressure sensor and affixed to a surgical glove. Then, during an initial palpation of a subject, in responsive to a contacting of the glove to a portion of a subject body, pressure data is sensed in the e-tattoo is wirelessly transmitted to a remote computing device where the pressure data is recorded in connection with when the data is acquired. Optionally, audio or video reaction data by the subject is acquired contemporaneously with the pressure data and also stored in the remote computing device. Subsequently, during a different palpation of the same subject, in responsive to a contacting of the glove to the same portion of the subject body, pressure data once again is sensed in the e-tattoo and wirelessly transmitted to the remote computing device along with contemporaneously acquired audio or video reaction data by the subject. The pressure data is then compared for both palpations and presented visually in the remote computer. As well, a data store of pressure data with corresponding diagnostic notations is consulted with the pressure data so as to cognitively identify potential diagnostic information for presentation in the remote computer.

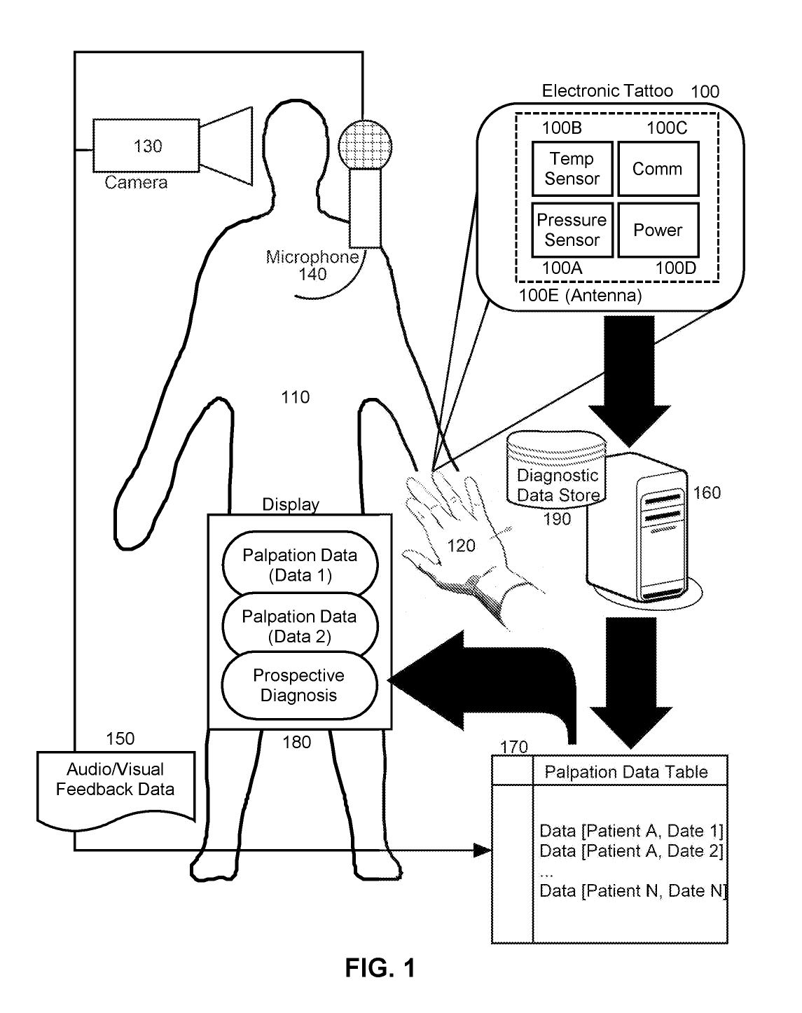

In further illustration, FIG. 1 pictorially shows a process for cognitive analysis of e-tattoo acquired palpation measurements. As shown in FIG. 1, a palpation of a patient 110 is performed by a health care provider utilizing a surgical glove 120 onto which an electronic tattoo 110 is affixed. The electronic tattoo, or e-tattoo, 100 includes a pressure sensor, a temperature sensor 100B, communications circuitry 100C and a power source 100D along with an antenna 100E. As the health care provider presses the surgical glove 120 to a portion of the body of the patient 110, pressure data from the pressure sensor 100A, and optionally temperature data from the temperature sensor 100B, is wireless transmitted by communications circuitry 100C through the antenna 100E to a host computing system 160. The data is then stored in a palpation data table 170 in connection with a date and/or time when the data was collected and an identity of the patient 110. As well, a physical reaction of the patient 110 is recorded either visually by video camera 130, audibly by microphone 140 or both. The resultant audio/visual feedback data 150 is then also stored in the palpation data table 170 in connection with the collected data.

Subsequently, a different palpation is performed in connection with the same patient 110, either by the same health care provider or a different healthy care provider. In the different palpation, a surgical glove 120 outfitted with an e-tattoo 100 again is utilized to collect data such as pressure data and temperature data and the collected data is stored in the palpation data table 170 in connection with a data and/or time when the data was collected along with the identity of the patient 110. As well, once again a physical reaction of the patient 110 is recorded either visually by video camera 130, audibly by microphone 140 or both. The resultant audio/visual feedback data 150 again is also stored in the palpation data table 170 in connection with the collected data.

Finally, a display 180 is generated including both the collected data of the first palpation and also the collected data of the later palpation. The display may include the audio/visual feedback data so that the health care provider conducting the later palpation of the patient 110 may compare the reaction of the patient from the prior palpation to the reaction of the patient in the later palpation even if a different health care provider had conducted the prior palpation. As well, the collected data from the later palpation and the difference between the collected data from both palpations may be compared to previously collected data in a diagnostic data store 190 correlating pressure data with prospective diagnoses. To the extent that the pressure data from either or both palpations of the patient 110 matches a prospective diagnosis in the diagnostic data store 190, the prospective diagnosis also is presented in the display 180.

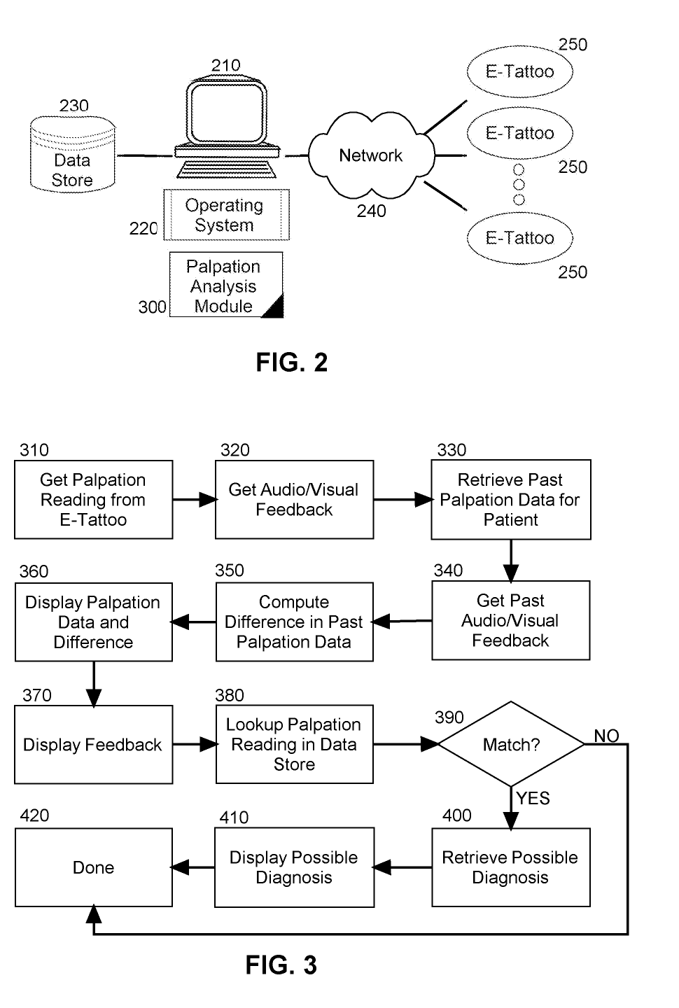

The process described in connection with FIG. 1 may be implemented in a data processing system. In yet further illustration, FIG. 2 schematically illustrates a data processing system configured for cognitive analysis of e-tattoo acquired palpation measurements. The system includes a host computing platform 210 that includes one or more computers, each with memory and at least one processor. The host computing platform 210 is adapted for communicative coupling to one or more e-tattoos 250 over a wireless communication network 240, each of the e-tattoos 250 transmitting pressure data over the wireless communications network 240 to the host computing platform for storage in a data store 230 coupled to the host computing platform 210.

An operating system 220 executes in the memory of the host computing platform 210. The operating system 220 supports the operation of a palpation analysis module 300. The palpation analysis module 300 includes program code that when executes in the memory of the host computing platform, is enabled to receive palpation data from one of the e-tattoos 250 during a contemporary palpation of a patient. As well, the program code is enabled to receive audio/visual feedback data acquired during the contemporary palpation of the patient. The program code then stores the palpation data and the audio/visual feedback data of the contemporary palpation of the patient in the data store 230 in connection with an identity of the patient.

Then, the program code retrieves from the data store 230 palpation data including any audio/visual feedback data for the same patient from a prior palpation conducted previously based upon the identity of the patient. The program code yet further matches the contemporary and prior palpation data to a prospective diagnosis stored in the data store 230. Finally, the program code presents in a display of the host computing platform 210 the prior palpation data, the contemporary palpation data, the audio/visual feedback data, and the prospective diagnosis, if any. Optionally, the program code presents in the display any threshold differences in the prior palpation data and the contemporary palpation data.

In even yet further illustration of the operation of the palpation analysis module 300, FIG. 3 is a flow chart illustrating a process for cognitive analysis of e-tattoo acquired palpation measurements. Beginning in block 310, palpation data is received from an e-tattoo contacting a portion of a body of a patient, for instance when a surgical glove of a health care provider to which the e-tattoo is affixed contacts the portion of the body of the patient. The palpation data includes pressure data which may be computationally correlated to a tenderness of human tissue at the portion of the body, a shape of a mass of material disposed at the portion of the body, or a movement of a muscular or skeletal structure at the portion of the body. Optionally, the palpation data may also include temperature data sensed at a point of contact of the e-tattoo to the portion of the body. Finally, either or both of audio and visual feedback acquired of the patient may be received at block 320 in connection with the receipt of the palpation data.

In block 330, palpation data collected previously from the same patient may be retrieved from a data store and in block 340, either or both of audio and visual feedback acquired of the patient at the time of the previous collection of the palpation data may be retrieved from the data store. In block 350, a difference is computed between the palpation data received contemporaneously and the palpation data previously collected so as to determine, for example, a change in tenderness of the portion of the body subject to the palpation, or a change in mass size at the portion of the body subject to the palpation. Finally, at block 360, the palpation data is displayed to the health care provider administering the palpation along with any computed difference. As well, in block 370, either or both of the audio or visual feedback from either or both of the prior or contemporaneous palpations is displayed to the health care provider.

In block 380, the contemporaneously received palpation data is used as a key to lookup a prospective diagnosis in a data store. In decision block 390, it is determined if any prospective diagnosis matches the contemporaneously received palpation data. If so, in block 400, a prospective diagnosis is retrieved that matches the contemporaneously received palpation data. Then, in block 410, the prospective diagnosis also is displayed to the health care provider administering the contemporaneous palpation of the patient. Finally, the process ends in block 420.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method of cognitive analysis of e-tattoo acquired palpation measurements, the method comprising:
receiving pressure data from an e-tattoo affixed to surgical gloves in connection with a contemporaneous palpation of a patient and storing the received pressure data in a data store;
retrieving from the data store previously stored pressure data also received from a different e-tattoo affixed to different surgical gloves in connection with a previous palpation of the patient; and,
generating in a display of a computer a visualization of a comparison of the pressure data both received and previously stored.

2. The method of claim 1, further comprising acquiring video imagery of the patient during the contemporaneous palpation.

3. The method of claim 2, further comprising:
additionally retrieving from the data store previously stored video imagery of the patient during the previous palpation; and,
presenting in the display the previously stored video imagery.

4. The method of claim 1, further comprising acquiring audio of the patient during the contemporaneous palpation.

5. The method of claim 4, further comprising:
additionally retrieving from the data store previously stored audio of the patient during the previous palpation; and,
playing back through the computer the previously stored video imagery.

6. The method of claim 1, further comprising:
locating in the data store a diagnostic notation corresponding to the received pressure data during the contemporaneous palpation; and,
displaying in the display the located diagnostic notation.
